(19) United States
(12) Patent Application Publication
(10) Pub. No.: US 2018/0263889 A1
(43) Pub. Date: Sep. 20, 2018

(54) COMPOSITION COMPRISING STABILIZED POLYMER PARTICLES AND A NONIONIC SURFACTANT

(71) Applicant: L'OREAL, Paris (FR)

(72) Inventors: Philippe ILEKTI, Chevilly Larue (FR); Alexandra SCHVENT, Chevilly Larue (FR)

(21) Appl. No.: 15/537,423

(22) PCT Filed: Nov. 18, 2015
(86) PCT No.: PCT/EP2015/080619

§ 371 Date: Jun. 18, 2017

(30) Foreign Application Priority Data
Dec. 18, 2014 (FR) 1462728

(51) Int. Cl. A61K 8/81; A61K 8/86; A61K 8/92; A61K 8/04; A61Q 1/10

(52) U.S. Cl. CPC A61K 8/8152; A61K 8/86; A61K 8/8111; A61K 2800/52; A61K 8/92; A61K 8/04; A61Q 1/10; A61K 8/8117

(57) ABSTRACT

The present invention relates to a composition, especially a cosmetic composition, for caring for and/or making up keratin fibres, and more particularly the eyelashes, comprising at least: a non-aqueous medium containing at least one hydrocarbon-based oil, particles of at least one polymer surface-stabilized with a stabilizer, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4; and a nonionic surfactant. The present invention also relates to a cosmetic process for making up and/or caring for keratin fibres, and also to the use of a dispersion of particles of at least one polymer surface-stabilized with a stabilizer in a non-aqueous medium containing at least one hydrocarbon-based oil, for preparing a mascara composition.

The present invention is directed towards proposing compositions, especially cosmetic compositions, with improved persistence over time, which in particular have increased water resistance but are nevertheless easy to remove. The invention also relates more particularly to the field of caring for and/or making up keratin materials, especially keratin fibres.

The term “keratin materials” preferably means human keratin materials, especially keratin fibres.

The present invention proves to be most particularly advantageous for caring for and/or making up keratin fibres.

The term “keratin fibres” in particular means the eyelashes and/or the eyebrows, and preferably the eyelashes. For the purposes of the present invention, this term “keratin fibres” also extends to synthetic false eyelashes.

The mascara formulations, intended for caring for and/or making up keratin fibres and more particularly the eyelashes, which are the most widely used for ensuring good persistence on the eyelashes, are “waterproof” formulations, i.e. formulations that have good water resistance. They thus have a very low water content, or even are advantageously anhydrous. Such a formulation is usually a dispersion of at least one oily structuring agent, which may be a wax, a polymer, in particular a semi-crystalline polymer, or a lipophilic gelling agent in a non-aqueous solvent medium.

Unfortunately, these mascara formulations, which are appreciated for their level of persistence over time, prove, on the other hand, to be difficult to remove, especially with the usual makeup removers, which are mainly water-based or water-soluble. Their removal thus requires as a general rule the use of specific makeup removers based on oils or organic solvents. However, these makeup removers may leave an uncomfortable greasy residual film or an unaesthetic black deposit on the skin around the eyes (eyelids).

The need thus remains for compositions, especially cosmetic compositions, which are suitable for making up and/or caring for keratin fibres and in particular the eyelashes, which have properties in terms of persistence that are at least equivalent to those manifested by waterproof mascara formulations, but which are, on the other hand, compatible with easy makeup removal that does not leave any black marks on the skin.

Contrary to all expectation, the inventors have found that the presence of at least one hydrocarbon-based oil as defined below, of at least specific particles of at least one stabilized polymer as defined below and of at least one nonionic surfactant as defined below makes it possible precisely to satisfy this need.

Thus, according to one of its aspects, the present invention relates to a composition, especially a cosmetic composition, for making up and/or caring for keratin fibres, and more particularly the eyelashes, comprising at least:
a non-aqueous medium containing at least one hydrocarbon-based oil,
particles of at least one polymer that is surface-stabilized with a stabilizer, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4; and
a nonionic surfactant.

According to an advantageous variant, said particles are in dispersion in said non-aqueous medium containing at least one hydrocarbon-based oil.

Contrary to all expectation, and as emerges from the examples given below, a composition according to the invention makes it possible to gain access to a mascara formulation, which is preferably anhydrous, which has expected properties in terms of water resistance but which is advantageously easy to remove by virtue of the combination of at least one hydrocarbon-based oil as defined below, of at least specific particles of at least one stabilized polymer as defined below and of a specific nonionic surfactant as defined below.

The compositions according to the invention may especially be makeup compositions intended for affording the desired makeup effect, by their use alone on the eyelashes, but may also be non-pigmented or coloured compositions intended either to be superimposed on a makeup already deposited on the eyelashes or to be coated with a related makeup film: they are then termed, respectively, a top coat or a base coat. They may also be compositions intended for affording only care on keratin fibres and in particular the eyelashes.

According to another of its aspects, a subject of the invention is also a process, especially a cosmetic process, for making up and/or caring for keratin fibres, especially the eyelashes, comprising at least one step which consists in applying to said keratin fibres a composition in accordance with the invention.

The present invention is also directed towards the use of a dispersion of particles of at least one polymer that is surface-stabilized with a stabilizer in a non-aqueous medium containing at least one hydrocarbon-based oil, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4 for preparing a mascara composition.

For the purposes of the invention, the term “easy to remove” or “easy makeup removal” means makeup removal obtained with a small number of cotton pads by using standard waterproof makeup removers (two-phase, makeup-removing oils, wipes or hot water) and/or which does not leave any marks on the skin.

Nonionic Surfactant

As stated previously, a composition according to the invention comprises at least one nonionic surfactant.

A nonionic surfactant that is suitable for use in the invention is a hydrocarbon-based compound.

Advantageously, a nonionic surfactant that is suitable for use in the invention is a nonionic surfactant with an HLB (hydrophilic-lipophilic balance) value of greater than or equal to 8 at 25° C.

The term hydrophilic/lipophilic balance is well known to those skilled in the art and represents a characteristic magnitude of a surfactant, which is proportionately greater the higher the solubility in water of the surfactant.

For the nonionic surfactants that are suitable for use in the invention, use will be made of the calculation method defined in the publication by W. C. Griffin, J. Soc. Cosm. Chem. 1954 (Vol. 5), pages 249-256, namely HLB=20×Mh/M, in which formula Mh is the molecular mass of the hydrophilic portion of the molecule and M is the total molecular mass of the molecule, giving a result on a scale from 0 to 20. For example, an HLB value of 0 calculated according to the Griffin method corresponds to a completely lipophilic/hydrophobic molecule, and a value of 20 corresponds to a completely hydrophilic/lipophobic molecule.

Among the nonionic surfactants that are suitable for use in the invention with an HLB of greater than or equal to 8 at 25° C., used alone or as a mixture, mention may be made especially of:
oxyethylenated ethers (which may comprise from 10 to 150 oxyethylene groups) of glycerol;
oxyethylenated ethers (which may comprise from 10 to 1000 oxyethylene groups) of fatty alcohols (especially of a C₈-C₂₄ and preferably C₁₂-C₁₈ alcohol) such as the oxyethylenated ether of cetearyl alcohol containing 30 oxyethylene groups (CTFA name: Ceteareth-30), the oxyethylenated ether of stearyl alcohol containing 20 oxyethylene groups (CTFA name: Steareth-20) such as Brij 78 sold by the company Uniqema or Brij s 020-PA-(SG) sold by the company Croda, and the oxyethylenated ether of cetearyl alcohol containing 30 oxyethylene groups (CTFA name: Ceteareth-30),
fatty acid esters (especially of a C₈-C₂₄ and preferably C₁₆-C₂₂ acid) of polyethylene glycol (which may comprise from 10 to 150 ethylene glycol units), such as PEG-50 stearate and PEG-40 monostearate sold especially under the name Myrj 52P® by the company ICI Uniqema,
fatty acid esters (especially of a C₈-C₂₄ and preferably C₁₆-C₂₂ acid) of oxyethylenated glyceryl ethers (which may comprise from 10 to 150 oxyethylene groups), for instance PEG-200 glyceryl monostearate sold especially under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt,
fatty acid esters (especially of a C₈-C₂₄ and preferably C₁₆-C₂₂ acid) of oxyethylenated sorbitol ethers (which may comprise from 10 to 150 oxyethylene groups), for instance polysorbate 60 sold under the name Tween 60® by the company Uniqema,
dimethicone copolyol, such as the product sold under the name Q2-5220® by the company Dow Corning,
dimethicone copolyol benzoate (sold especially under the name Finsolv SLB 101® and 201® by the company Finetex),
copolymers of propylene oxide and of ethylene oxide, also known as EO/PO polycondensates,
and mixtures thereof.

EO/PO polycondensates are more particularly copolymers formed from polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,

in which a ranges from 10 to 120 and b ranges from 1 to 100.

The EO/PO polycondensate preferably has a weight-average molecular weight ranging from 1000 to 15 000 and better still ranging from 2000 to 13 000. Advantageously, said EO/PO polycondensate has a cloud point, at 10 g/l in distilled water, of greater than or equal to 20° C., preferably of greater than or equal to 60° C. The cloud point is measured according to the standard ISO 1065. As EO/PO polycondensate that may be used according to the invention, mention may be made of the polyethylene glycol/polypropylene glycol/polyethylene glycol triblock polycondensates sold especially under the name Synperonic®, such as Synperonic PE/L44® and Synperonic PE/F127® by the company ICI.

Preferably, a nonionic surfactant that is suitable for use in the invention is chosen from oxyethylenated ethers (which may comprise from 10 to 1000 oxyethylene groups) of fatty alcohols (especially of a C₈-C₂₄ and preferably C₁₂-C₁₈ alcohol) such as the oxyethylenated ether of stearyl alcohol containing 30 oxyethylene groups (CTFA name: Ceteareth-30), the oxyethylenated ether of stearyl alcohol containing 20 oxyethylene groups (CTFA name: Steareth-20) such as Brij 78 sold by the company Uniqema or Brij s20-PA-(SG) sold by the company Croda, and the oxyethylenated ether of cetearyl alcohol containing 30 oxyethylene groups (CTFA name: Ceteareth-30).

More preferentially, a nonionic surfactant that is suitable for use in the invention is an oxyethylenated ether of fatty alcohols, also known as ethoxylated fatty alcohol.

Even more preferentially, a nonionic surfactant that is suitable for use in the invention is the oxyethylenated ether of stearyl alcohol containing 20 oxyethylene groups. A composition according to the invention may comprise from 1% to 15% by weight, preferably from 1% to 8% by weight and even more preferentially from 1.5% to 7% by weight of nonionic surfactant(s) relative to the total weight of the composition.

### Hydrocarbon-Based Oil

The composition according to the invention comprises a hydrocarbon-based oil.

This oil may be volatile (vapour pressure greater than or equal to 0.13 Pa measured at 25° C.) or non-volatile (vapour pressure less than 0.13 Pa measured at 25° C.).

Preferably, the hydrocarbon-based oil is volatile.

The hydrocarbon-based oil is an oil (non-aqueous compound) that is liquid at room temperature (25° C.).

The term “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

The hydrocarbon-based oil may be chosen from:

hydrocarbon-based oils containing from 8 to 16 carbon atoms, and especially:
branched C₈-C₁₅ alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade name Isopar or Permethyl,
linear alkanes, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C₁₃) obtained in Examples 1 and 2 of patent application WO 2008/155 059 from the company Cognis, and mixtures thereof,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have chain lengths varying from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quina oil, rye oil, safflower oil, candlenut oil, passion-flower oil and musk rose oil; shea butter; or else caprylic/capric acid triglycerides, for instance those sold by the company Stearineries Dubois or those sold under the names Miglyol 810®, 812® and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
synthetic esters such as oils of formula R₁COOR₂ in which R₁ represents a linear or branched fatty acid residue containing from 1 to 40 carbon atoms and R₂ represents an, in particular, branched hydrocarbon-based chain containing from 1 to 40 carbon atoms, on condition that R₁+R₂≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance oetyldodecanol, isostearyl alcohol, oleyl alcohol, 2-hexyldecanol, 2-butyloctanol and 2-undecylpentadecanol,
mixtures thereof.

More particularly, the content of hydrocarbon-based oil(s) ranges from 30% to 75% by weight and preferably from. 40% to 60% by weight relative to the total weight of the composition.

This hydrocarbon-based oil may be provided totally or partly with the surface-stabilized polymer particles, in particular when these particles are introduced into the composition in the form of a pre-prepared dispersion of stabilized polymer particles. In this case, the hydrocarbon-based oil present in the composition represents at least the non-aqueous medium of the dispersion of polymer particles.

Advantageously, the hydrocarbon-based oil is apolar (thus formed solely from carbon and hydrogen atoms).

The hydrocarbon-based oil is preferably chosen from hydrocarbon-based oils containing from 8 to 16 carbon atoms and better still from 12 to 16 carbon atoms, in particular the apolar oils described previously.

Preferentially, the hydrocarbon-based oil is isododecane. More particularly, the isododecane content ranges from 30% to 75% by weight and preferably from 40% to 60% by weight relative to the total weight of the composition.

Preferably, the hydrocarbon-based oil(s), in particular isododecane, constitute the only oil(s) of the composition, or are present in a predominant weight content relative to the additional oil(s) that may be present in the composition.

Thus, according to a particular embodiment, the hydrocarbon-based oil(s) are present in a composition according to the invention in a content ranging from 30% to 75% by weight and preferably from 40% to 60% by weight relative to the total weight of the composition, the hydrocarbon-based oil(s) preferably being apolar, more preferentially volatile, even more preferentially containing from 8 to 16 carbon atoms, or even better still isododecane.

In accordance with a particular embodiment of the invention, if the composition contains one or more non-volatile oils, their content advantageously does not exceed 10% by weight, preferably does not exceed 5% by weight relative to the total weight of the composition, and better still does not exceed 2% by weight relative to the total weight of the composition, or even is free of non-volatile oil(s).

### Polymer Particles

The composition according to the invention moreover comprises particles, which are generally spherical, of at least one surface-stabilized polymer.

Preferably, the particles are introduced into the composition in the form of a dispersion of particles, which are generally spherical, of at least one surface-stabilized polymer, in an oily medium, advantageously containing at least one hydrocarbon-based oil, as defined previously.

The polymer of the particles is a C₁-C₄ alkyl (meth)acrylate polymer.

The C₁-C₄ alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate.

A C₁-C₄ alkyl acrylate monomer is advantageously used. Preferentially, the polymer of the particles is a methyl acrylate and/or ethyl acrylate polymer.

The polymer of the particles may also comprise an ethylenically unsaturated acid monomer or the anhydride thereof, chosen especially from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof.

Preferably, the ethylenically unsaturated acid monomer is chosen from (meth)acrylic acid, maleic acid and maleic anhydride.

The salts may be chosen from salts of alkali metals, for example sodium or potassium; salts of alkaline-earth metals, for example calcium, magnesium or strontium; metal salts, for example zinc, aluminium, manganese or copper; ammonium salts of formula NH₄⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles may thus comprise or consist essentially of 80% to 100% by weight of C₁-C₄ alkyl (meth)acrylate and of 0 to 20% by weight of ethylenically unsaturated acid monomer, relative to the total weight of the polymer.

According to a first embodiment of the invention, the polymer consists essentially of a polymer of one or more C₁-C₄ alkyl (meth)acrylate monomers.

According to a second embodiment of the invention, the polymer consists essentially of a copolymer of C₁-C₄ (meth)acrylate and of (meth)acrylic acid or maleic anhydride.

The polymer of the particles may be chosen from:

methyl acrylate homopolymers

ethyl acrylate homopolymers

methyl acrylate/ethyl acrylate copolymers

methyl acrylate/ethyl acrylate/acrylic acid copolymers

methyl acrylate/ethyl acrylate/maleic anhydride copolymers

methyl acrylate/acrylic acid copolymers

ethyl acrylate/acrylic acid copolymers

methyl acrylate/maleic anhydride copolymers

ethyl acrylate/maleic anhydride copolymers.

Advantageously, the polymer of the particles is a non-crosslinked polymer.

The polymer of the particles preferably has a number-average molecular weight ranging from 2000 to 10 000 000 and preferably ranging from 150 000 to 500 000.

In the case of a particle dispersion, the polymer of the particles may be present in the dispersion in a content ranging from 21% to 58.5% by weight and preferably ranging from 36% to 42% by weight, relative to the total weight of the dispersion.

The stabilizer is an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4, preferably greater than 4.5 and even more advantageously greater than or equal to 5. Advantageously, said weight ratio ranges from 4.5 to 19, preferably from 5 to 19 and more particularly from 5 to 12.

Thus, according to a particular embodiment, a composition according to the invention comprises one or more stabilizers, said stabilizer(s) being a statistical copolymer of isobornyl (meth)acrylate and of C₁-C₄ alkyl (meth)acrylate present in an isobornyl (meth)acrylate/C₁-C₄ alkyl (meth)acrylate weight ratio of greater than or equal to 5.

Advantageously, the stabilizer is chosen from:

isobornyl acrylate homopolymers

statistical copolymers of isobornyl acrylate/methyl acrylate

statistical copolymers of isobornyl acrylate/methyl acrylate/ethyl acrylate

statistical copolymers of isobornyl methacrylate/methyl acrylate

in the weight ratio described previously.

The stabilizing polymer preferably has a number-average molecular weight ranging from 10 000 to 400 000 and preferably ranging from 20 000 to 200 000.

The stabilizer is in contact with the surface of the polymer particles and thus makes it possible to stabilize these particles at the surface, in particular in order to keep these particles in dispersion in the non-aqueous medium of the dispersion.

Advantageously, the combination of the stabilizer(s)+polymer(s) of the particles present in particular in the dispersion comprises from 10% to 50% by weight of polymerized isobornyl (meth)acrylate and from 50% to 90% by weight of polymerized C₁-C₄ alkyl (meth)acrylate, relative to the total weight of the combination of the stabilizer(s) polymer(s) of the particles.

Preferentially, the combination of the stabilizer(s)+polymer(s) of the particles present in particular in the dispersion comprises from 15% to 30% by weight of polymerized isobornyl (meth)acrylate and from 70% to 85% by weight of polymerized C₁-C₄ alkyl (meth)acrylate, relative to the total weight of the combination of the stabilizer(s) polymer(s) of the particles.

Preferably, the stabilizer(s) are soluble in the hydrocarbon-based oil, in particular soluble in isododecane.

According to a theory which should not limit the scope of the present invention, the inventors put forward the hypothesis that the surface stabilization of the C₁-C₄ alkyl (meth)acrylate polymer particles results from a phenomenon of surface adsorption of the stabilizer onto the C₁-C₄ alkyl (meth)acrylate polymer particles.

When the polymer particles are provided in the composition in the form of a pre-prepared dispersion, the oily medium of this polymer dispersion comprises a first hydrocarbon-based oil. Reference may be made to that which has been indicated previously concerning this oil as regards its nature.

Advantageously, the hydrocarbon-based oil is apolar and preferably chosen from hydrocarbon-based oils containing from 8 to 16 carbon atoms, in particular the apolar oils described previously.

Preferentially, the hydrocarbon-based oil is isododecane.

The polymer particles, in particular in the dispersion, preferably have an average size, especially a number-average size, ranging from 50 to 500 nm, especially ranging from 75 to 400 nm and better still ranging from 100 to 250 nm.

In general, a dispersion of polymer particles that is suitable for use in the invention may be prepared in the following manner, which is given as an example.

The polymerization may be performed in dispersion, i.e. by precipitation of the polymer during formation, with protection of the formed particles with a stabilizer.

In a first step, the stabilizing polymer is prepared by mixing the constituent monomer(s) of the stabilizing polymer, with a radical initiator, in a solvent known as the synthesis solvent, and by polymerizing these monomers. In a second step, the constituent monomer(s) of the polymer of the particles are added to the stabilizing polymer formed and polymerization of these added monomers is performed in the presence of the radical, initiator.

When the non-aqueous medium is a non-volatile hydrocarbon-based oil, the polymerization may be performed in an apolar organic solvent (synthesis solvent), followed by adding the non-volatile hydrocarbon-based oil (which should be miscible with said synthesis solvent) and selectively distilling off the synthesis solvent.

A synthesis solvent which is such that the monomers of the stabilizing polymer and the free-radical initiator are soluble therein, and the polymer particles obtained are insoluble therein, so that they precipitate therein during their formation, is thus chosen.

In particular, the synthesis solvent may be chosen from alkanes such as heptane or cyclohexane.

When the non-aqueous medium is a volatile hydrocarbon-based oil, the polymerization may be performed directly in said oil, which thus also acts as synthesis solvent. The monomers should also be soluble therein, as should the free-radical initiator, and the polymer of the particles obtained should be insoluble therein.

The monomers are preferably present in the synthesis solvent, before polymerization, in a proportion of 5-20% by weight. The total amount of monomers may be present in the solvent before the start of the reaction, or part of the monomers may be added gradually as the polymerization reaction proceeds.

The free-radical initiator may especially be azobisisobutyronitrile or tert-butyl peroxy-2-ethylhexanoate.

The polymerization may be performed at a temperature ranging from 70 to 110° C.

The polymer particles are surface-stabilized, when they are formed during the polymerization, by means of the stabilizer.

The stabilization may be performed by any known means, and in particular by direct addition of the stabilizer, during the polymerization.

The stabilizer is preferably also present in the mixture before polymerization of the monomers of the polymer of the particles. However, it is also possible to add it continuously, especially when the monomers of the polymer of the particles are also added continuously.

From 10% to 30% by weight and preferably from 15% to 25% by weight of stabilizer may be used relative to the total weight of monomers used (stabilizer+polymer of the particles).

The polymer particle dispersion advantageously comprises from 30% to 65% by weight and preferably from 40% to 60% by weight of solids, relative to the total weight of the dispersion.

Moreover, the composition according to the invention advantageously comprises a content of stabilized polymer particles, described previously, of beₜween 5% and 40% by weight, more particularly from 8% to 30% by weight and preferably from 10% to 25% by weight, relative to the total weight of the composition (content expressed as solids).

Moreover, the composition according to the invention advantageously comprises a content of dispersion, described previously, of between 10% and 60% by weight, more particularly from 15% to 45% by weight, relative to the total weight of the composition.

### Aqueous Phase

A composition according to the invention may comprise an aqueous phase.

The aqueous phase may comprise water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at room temperature.

Such polyols may be used in a proportion ranging from 0.2% to 10% by weight, preferably from 0.5% to 8% by weight and even more preferentially from 0.5% to 6% by weight of C2-C₃2 polyol relative to the total weight of the composition.

The polyols that are advantageously suitable for the formulation of a composition according to the present invention are those especially containing from 2 to 32 carbon atoms, preferably from 3 to 16 carbon atoms and in particular from 3 to 7 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof, in particular pentylene glycol.

According to a preferred embodiment of the invention, said polyol is chosen from ethylene glycol, pentaerythritol, trimethylolpropane, propylene glycol, pentylene glycol, glycerol, polyglycerols and polyethylene glycols, and mixtures thereof.

According to a particular mode, the composition of the invention may comprise at least pentylene glycol.

According to one particular embodiment, a composition according to the invention is anhydrous.

For the purposes of the invention, a composition is preferably anhydrous. More particularly, a composition according to the invention comprises less than 10% by weight of water, better still less than 5% by weight of water, in particular less than 2% by weight of water, or even less than 0.5% by weight of water relative to the total weight of the composition, and is especially free of water. Where appropriate, such small amounts of water may especially be introduced by ingredients of the composition that may contain residual amounts thereof.

A composition according to the invention advantageously comprises at least one gelling and/or structuring agent for the oil(s).

This (these) compounds may be chosen especially from lipophilic gelling agents, for instance hydrophobic-modified clays, such as modified magnesium silicate (Bentone gel VS38 from Rheox), hectorite modified with distearyldimethylammonium chloride (CTFA name: disteardimonium hectorite) sold especially under the name Bentone 38 CE by the company Rheox or under the name Bentone 38 VCG® by the company Elementis. These compounds may also be chosen from gums, such as silicone gums (dimethiconol); silicone elastomers, for instance the products sold under the KSG names by the company Shin-Etsu, under the name Trefil by the company Dow Corning or under the Gransil names by the company Grant Industries; and mixtures thereof

### Waxes

A composition according to the invention comprises at least one wax.

For the purposes of the present invention, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and preferably having an anisotropic crystal organization in the solid state. By bringing the wax to its melting point, it is possible to disperse it in the oil(s) and to form a macroscopically homogeneous mixture, and on returning the temperature of the mixture to room temperature, homogeneous recrystallization of the wax from the oil(s) of the mixture is obtained.

The waxes that may be used in the invention may be hydrocarbon-based waxes and/or silicone waxes, and may be of plant, mineral, animal and/or synthetic origin, preferably hydrocarbon-based. In particular, they have a melting point of greater than 40° C. and better still greater than 45° C., for example ranging from 50° C. to 110° C.

As waxes that may be used in the invention, mention may be made of those generally used in cosmetics: they are especially of natural origin, such as beeswaxes, carnauba wax, such as the product sold especially under the name Cerauba T1 by the company Baerlocher, candelilla wax, ouricury wax, Japan wax, cork fibre wax or sugarcane wax, rice wax, montan wax, paraffin waxes especially such as the product sold under the name Affine 56-58 Pastilles by the company Baerlocher, lignite wax or microcrystalline wax, ceresin or ozokerite, hydrogenated waxes such as jojoba oil; synthetic waxes such as polyethylene waxes derived from the polymerization or copolymerization of ethylene and Fischer-Tropsch waxes, or alternatively fatty acid esters such as octacosanyl stearate, glycerides that are concrete at 40° C. and better still at 45° C., silicone waxes such as alkyl or alkoxy dimethicones with an alkyl or alkoxy chain of 10 to 45 carbon atoms, poly(di)methylsiloxane esters that are solid at 40° C., the ester chain of which comprises at least 10 carbon atoms; and mixtures thereof.

A composition in accordance with the invention comprises a content of wax(es) of greater than or equal to 5% by weight and better still 10% by weight relative to the total weight of the composition.

As a guide, a composition according to the invention may comprise from 0.01% to 50% by weight, preferably from 2% to 40% by weight, better still from 5% to 35% by weight and better still from 10% to 30% by weight of wax(es), relative to the total weight of the composition.

Thus, according to a particular embodiment, a composition according to the invention also comprises at least one wax, the wax(es) being present in a content of greater than or equal to 5% by weight, in particular ranging from 10% to 30% by weight, relative to the total weight of the composition.

### Pasty Compound

For the purposes of the present invention, the term “pasty compound” is intended to denote a fatty compound with a reversible solid/liquid change of state, and comprising at a temperature of 25° C. a liquid fraction and a solid fraction.

A pasty compound is advantageously chosen from:
lanolin and derivatives thereof,
petroleum jelly,
polyol ethers,
polymeric or non-polymeric silicone compounds,
vinyl polymers, especially:
   olefin homopolymers and copolymers,
   hydrogenated diene homopolymers and copolymers,
   linear or branched homopolymer or copolymer oligomers of alkyl (meth)acrylates preferably containing a C₈-C₃₀ alkyl group,
   homopolymer and copolymer oligomers of vinyl esters containing C₈-C₃₀ alkyl groups,
   homopolymer and copolymer oligomers of vinyl ethers containing C₈-C₃₀ alkyl groups,
liposoluble polyethers resulting from polyetherification between one or more C₂-C₁₀₀ and preferably C₂-C₅₀ diols,
esters such as esters of a glycerol oligomer, arachidyl propionate, phytosterol esters, fatty acid triglycerides and derivatives thereof, pentaerythritol esters, esters of a diol dimer and a diacid dimer, mango butter, shea butter, and mixtures thereof, and
mixtures thereof.

As a guide, a composition according to the invention contains an amount of pasty fatty substances of less than 10% by weight, or even less than 5% by weight, relative to the total weight of the composition, or even is free of pasty fatty substances.

### Dyestuffs

The compositions in accordance with the invention may comprise at least one dyestuff.

This (or these) dyestuff(s) are preferably chosen from pulverulent substances, liposoluble dyes and water-soluble dyes, and mixtures thereof.

Preferably, the compositions according to the invention comprise at least one pulverulent dyestuff. The pulverulent dyestuffs may be chosen from pigments and nacres, and preferably from pigments.

The pigments may be white or coloured, mineral and/or organic, and coated or uncoated. Among the mineral pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides. More preferentially, the pigments contained in the compositions according to the invention are chosen from iron oxides, such as especially those sold under the name Sunpuro Black Iron Oxide C33-7001® by the company Sun.

Thus, according to a particular embodiment, a composition according to the invention also comprises at least one dyestuff, the dyestuff(s) preferably being chosen from pulverulent materials, in particular pigments, more particularly from metal oxides such as iron oxides.

These dyestuffs may be present in a content ranging from 0.01% to 30% by weight relative to the total weight of the composition and in particular from 1% to 22% by weight relative to the total weight of the composition.

Preferably, the dyestuff(s) are chosen from one or more metal oxides that are present in a content of greater than or equal to 1% by weight relative to the total weight of the composition, and advantageously inclusively between 3% and 22% by weight relative to the total weight of the composition.

### Additives

The compositions according to the invention may also comprise any cosmetic active agent, such as active agents chosen from an additional volatile or non-volatile silicone oil, fillers, fibres, antioxidants, preserving agents, fragrances, bactericidal active agents, neutralizers, emollients, moisturizers, trace elements, softeners, sequestrants, acidifying or basifying agents, hydrophilic or lipophilic active agents, coalescers and vitamins, and mixtures thereof.

It is a matter of routine operations for a person skilled in the art to adjust the nature and the amount of the additives present in the compositions in accordance with the invention such that the desired cosmetic properties thereof are not thereby affected.

According to a preferred embodiment, a composition of the invention is in the form of a product for the eyelashes, in particular a mascara.

According to another embodiment, a composition of the invention may advantageously be in the form of a product for the eyebrows.

Preferably, a composition according to the invention is in the form of a composition for caring for and/or making up keratin fibres, in particular the eyelashes, preferably in the form of a mascara.

Such compositions are especially prepared according to the general knowledge of a person skilled in the art.

The composition according to the invention may be conditioned in a container delimiting at least one compartment that comprises said composition, said container being closed by a closing member.

The container may be in any suitable form. It may especially be in the form of a bottle, a tube, a jar or a case.

The closing member may be in the form of a removable stopper, a lid or a cover, especially of the type comprising a body fixed to the container and a cap articulated on the body. It may also be in the form of a member ensuring the selective closure of the container, especially a pump, a valve or a clapper.

The container may be combined with an applicator, especially in the form of a brush comprising an arrangement of bristles maintained by a twisted wire. Such a twisted brush is described especially in patent U.S. Pat. No. 4,887,622. It may also be in the form of a comb comprising a plurality of application members, obtained especially by moulding. Such combs are described, for example, in patent FR 2 796 529. The applicator may be in the form of a fine brush, as described, for example, in patent FR 2 722 380. The applicator may be in the form of a block of foam or of elastomer. The applicator may be free (sponge) or securely fastened to a rod borne by the closing member, as described, for example, in patent U.S. Pat. No. 5,492,426. The applicator may be securely fastened to the container, as described, for example, in patent FR 2 761 959.

The product may be contained directly in the container, or indirectly.

The closing member may be coupled to the container by screwing. Alternatively, the coupling between the closing member and the container is done other than by screwing, especially via a bayonet mechanism, by click-fastening or by gripping. The term “click-fastening” in particular means any system involving the crossing of a bead or cord of material by elastic deformation of a portion, especially of the closing member, followed by return to the elastically unconstrained position of said portion after the crossing of the bead or cord.

The container may be at least partially made of thermoplastic material. Examples of thermoplastic materials that may be mentioned include polypropylene or polyethylene.

The container may have rigid or deformable walls, especially in the fowl of a tube or a tube bottle.

The container may comprise means intended to bring about or facilitate the dispensing of the composition. By way of example, the container may have deformable walls so as to allow the composition to exit in response to a positive pressure inside the container, this positive pressure being caused by elastic (or non-elastic) squeezing of the walls of the container.

The container may be equipped with a drainer arranged in the region of the aperture of the container. Such a drainer makes it possible to wipe the applicator and possibly the rod to which it may be securely fastened. Such a wiping element is described, for example, in patent FR 2 792 618.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

In the description and the examples, the percentages are percentages by weight, unless otherwise indicated. The percentages are thus given on a weight basis relative to the total weight of the composition. The ingredients are mixed in the order and under the conditions that are easily determined by those skilled in the art.

### I. EXAMPLES OF PREPARATION OF DISPERSIONS

### Example 1

In a first step, 1300 g of isododecane, 337 g of isobornyl acrylate, 28 g of methyl acrylate and 3.64 g of tert-butyl peroxy-2-ethylhexanoate (Trigonox 21S from Akzo) were placed in a reactor. The isobornyl acrylate/methyl acrylate mass ratio is 92/8. The mixture was heated at 90° C. under argon with stirring.

After 2 hours of reaction, 1430 g of isododecane were added to the reactor feedstock and the mixture was heated to 90° C.

In a second step, a mixture of 1376 g of methyl acrylate, 1376 g of isododecane and 13.75 g of Trigonox 21S were run in over 2 hours 30 minutes, and the mixture was left to react for 7 hours. 3.3 litres of isododecane were then added and part of the isododecane was evaporated off to obtain a solids content of 50% by weight.

A dispersion of methyl acrylate particles stabilized with a statistical copolymer stabilizer containing 92% isobornyl acrylate and 8% methyl acrylate in isododecane was obtained.

The oily dispersion contains in total (stabilizer+particles) 80% methyl acrylate and 20% isobornyl acrylate.

The polymer particles of the dispersion have a number-average size of about 160 nm.

The dispersion is stable after storage for 7 days at room temperature (25° C.).

### Example 2

A dispersion of polymer in isododecane was prepared according to the preparation method of Example 1, using:

Step 1: 275.5 g of isobornyl acrylate, 11.6 g of methyl acrylate, 11.6 g of ethyl acrylate, 2.99 g of Trigonox 21, 750 g of isododecane; followed by addition, after reaction, of 750 g of isododecane.

Step 2: 539.5 g of methyl acrylate, 539.5 g of ethyl acrylate, 10.8 g of Trigonox 21S, 1079 g of isododecane. After reaction, addition of 2 litres of isododecane and evaporation to obtain a solids content of 35% by weight.

A dispersion in isododecane of methyl acrylate/ethyl acrylate (50/50) copolymer particles stabilized with an isobornyl acrylate/methyl acrylate/ethyl acrylate (92/4/4) statistical copolymer stabilizer was obtained.

The oily dispersion contains in total (stabilizer+particles) 40% methyl acrylate, 40% ethyl acrylate and 20% isobornyl acrylate.

The dispersion is stable after storage for 7 days at room temperature (25° C.).

### Example 3

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 303 g of methyl acrylate, 776 g of ethyl acrylate, 157 g of acrylic acid, 11 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (24.5/62.8/12.7) copolymer particles stabilized with an isobornyl acrylate/methyl acrylate/ethyl acrylate (92/4/4) statistical copolymer stabilizer was obtained.

The oily dispersion contains in total (stabilizer+particles) 10% acrylic acid, 20% methyl acrylate, 50% ethyl acrylate and 20% isobornyl acrylate.

The dispersion is stable after storage for 7 days at room temperature (25° C.).

### Example 4

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 145 g of methyl acrylate, 934 g of ethyl acrylate, 157 g of acrylic acid, 12.36 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (11.7/75.6/12.7) copolymer particles stabilized with an isobornyl acrylate/methyl acrylate/ethyl acrylate (92/4/4) statistical copolymer stabilizer was obtained.

The oily dispersion contains in total (stabilizer+particles) 10% acrylic acid, 10% methyl acrylate, 60% ethyl acrylate and 20% isobornyl acrylate.

The dispersion is stable after storage for 7 days at room temperature (25° C.).

### Example 5

A dispersion of polymer in isododecane was prepared according to the preparation method of Example 1, using:

Step 1: 48 g of isobornyl acrylate, 2 g of methyl acrylate, 2 g of ethyl acrylate, 0.52 g of Trigonox 21, 57.6 g of isododecane, 38.4 g of ethyl acetate; followed by addition, after reaction, of 540 g of isododecane and 360 g of ethyl acetate.

Step 2: 98 g of methyl acrylate, 73 g of ethyl acrylate, 25 g of maleic anhydride, 1.96 g of Trigonox 21S, 50.4 g of isododecane and 33.60 g of ethyl acetate. After reaction, addition of 1 litre of an isododecane/ethyl acetate mixture (60/40 weight/weight) and total evaporation of the ethyl acetate and partial evaporation of the isododecane to obtain a solids content of 46.2% by weight.

A dispersion in isododecane of methyl acrylate/ethyl acrylate/maleic anhydride (50/37.2/12.8) copolymer particles stabilized with an isobornyl acrylate/methyl acrylate/ethyl acrylate (92/4/4) statistical copolymer stabilizer was obtained.

The oily dispersion contains in total (stabilizer+particles) 10% maleic anhydride, 30% methyl acrylate, 40% ethyl acrylate and 20% isobornyl acrylate.

The dispersion is stable after storage for 7 days at room temperature (25° C.).

### Example 6

A dispersion of polymer in isododecane was prepared according to the preparation method of Example 1, using:

Step 1: 48.5 g of isobornyl methacrylate, 4 g of methyl acrylate, 0.52 g of Trigonox 21, 115 g of isododecane; followed by addition, after reaction, of 80 g of isododecane.

Step 2: 190 g of methyl acrylate, 1.9 g of Trigonox 21S, 190 g of isododecane. After reaction, addition of 1 litre of isododecane and partial evaporation of the isododecane to obtain a solids content of 48% by weight.

A dispersion in isododecane of methyl acrylate polymer particles stabilized with an isobornyl methacrylate/methyl acrylate (92/8) statistical copolymer stabilizer was obtained.

The oily dispersion contains in total (stabilizer+particles) 80% methyl acrylate and 20% isobornyl methacrylate.

The dispersion is stable after storage for 7 days at room temperature (25° C.).

### II. COMPOSITION EXAMPLES: MASCARAS

Mascara formulations in accordance with the invention (compositions 1 and 2) or not in accordance with the invention (compositions 3 and 4) are prepared as described below.

The starting materials are first weighed out carefully using a balance (precision=0.01 g).

Preparation of Phase A

The ingredients of phase A are melted in a jacketed heating pan in which circulates an oil whose temperature is controlled by means of a thermostatic oil bath. The nominal temperature is set at 95-98° C.

Preparation of Phase B

Phase B is prepared at 70° C. with stirring for 30 minutes using a Rayneri blender.

Mixing of Phases A and B

After phase A has totally melted, phase B is poured into phase A with stirring for 30 minutes.

Addition of Phase C

Phase C is introduced into phase A+B. Stirring is continued for 10 minutes.

The preparation is cooled to room temperature.

Addition of Phase D

Phase D is introduced at room temperature with stirring until a homogeneous preparation is obtained.

End of Formulation

The mascara thus obtained is transferred into a closed container to prevent it from drying out on contact with air. After 24 hours, the satisfactory nature of the homogeneity and the dispersion of the pigment are evaluated.

| | | Composition 1 | Composition 2 | Composition 3 | Composition 4 |
|---|---|---|---|---|---|
| | | According to | According to | Outside the | Outside the |
| Phase | Compounds | the invention | the invention | invention | invention |
| Phase A | Carnauba wax | 5.0% | 5.0% | 5.0% | 5.0% |
| | (Cerauba T1 ® sold | | | | |
| | by the company | | | | |
| | Baerlocher) | | | | |
| | Paraffin (Affine 56- | 10.0% | 10.0% | 10.0% | 10.0% |
| | 58 Pastilles ® sold by | | | | |
| | the company | | | | |
| | Baerlocher) | | | | |
| | Steareth-20 (Brij | 2.0% | 6.0% | — | — |
| | S20-PA-(SG) ® sold | | | | |
| | by the company | | | | |
| | Croda) | | | | |
| Phase B | Isododecane sold by | Qs 100 | Qs 100 | Qs 100 | Qs 100 |
| | the company Ineos | | | | |
| | Disteardimonium | 2.0% | 2.0% | 2.0% | 2.0% |
| | hectorite (Bentone 38 | | | | |
| | VCG ® sold by the | | | | |
| | company Elementis) | | | | |
| | Propylene carbonate | 0.5% | 0.5% | 0.5% | 0.5% |
| | (Jeffsol Propylene | | | | |
| | Carbonate ® sold by | | | | |
| | the company | | | | |
| | Huntsman) | | | | |
| | (Methyl acrylate)-co- | 20.0% | 20.0% | 20.0% | — |
| | (isobornyl acrylate) | | | | |
| | (80.7/19.3) | | | | |
| | copolymer | | | | |
| | (80.7/19.3) dissolved | | | | |
| | in isododecane | | | | |
| | according to | | | | |
| | Preparation Example | | | | |
| | 1 described | | | | |
| | previously | | | | |
| Phase C | Iron oxides (Sunpuro | 7.0% | 7.0% | 7.0% | 7.0% |
| | C33-7001 ® sold by | | | | |
| | the company Sun) | | | | |
| Phase D | Preserving agent | 1.0% | 1.0% | 1.0% | 1.0% |

Evaluation of the Smudge Resistance and the Ease of Makeup Removal

| | Composition 1 | Composition 2 | Composition 3 | Composition 4 |
|---|---|---|---|---|
| | According to | According to | Outside the | Outside the |
| Compositions | the invention | the invention | invention | invention |
| Smudge resistance (/9) | 1 | 2 | 1 | 5 |
| Number of two-phase | 3 | 2 | >5 | 2 |
| makeup removal cotton | | | | |
| pads | | | | |

1/ Evaluation of the Smudge Resistance:

According to the present patent application, the term “smudge resistance” means the in vitro water resistance evaluated according to the following protocol:
the composition is applied to three specimens of 30-node straight Caucasian hair (60 eyelashes 1 cm long) with a fringe length of 2 cm, by making 3×10 passages at 2-minute intervals with reuptake of product between each series of 10. Each specimen is then dried at room temperature for a drying time of 1 hour.

The three made-up specimens are immersed in a container containing water at 20° C. for a given time (1 hour). The three specimens are then wiped to-and-fro five times with a Wypall L40 square swab from Kimberley Clark.
The presence of marks deposited by the specimen is then evaluated.
The resulting marks are attributed a score between 0 and 9; 0 being the score obtained when no marks are deposited by the specimen, and 9 the score obtained when very substantial marks are deposited.

2/ Evaluation of the Ease of Makeup Removal:

In order to evaluate the makeup-removing result of the composition according to the present patent application, an in vitro test is performed according to the following protocol:
the composition is applied to three specimens of 30-node straight Caucasian hair (60 eyelashes 1 cm long) with a fringe length of 2 cm, by making 3×10 passages at 2-minute intervals with reuptake of product between each series of 10.
Each specimen is then dried at room temperature for a drying time of 1 hour.
3 ml of a two-phase makeup remover are then deposited on a cotton support such as a Demakup® disk, and each cotton pad is then folded in two around its respective specimen.
A gentle pressure is applied to each cotton pad and said cotton pads are pulled relative to the specimens.
The deposit on each cotton pad is then observed visually. This operation is repeated until no more black marks are observed on the cotton pad.
The number of cotton pads required to remove the makeup from the specimen is then noted.

Thus, compositions 1 and 2 according to the invention give good smudge resistance, i.e. they make it possible to avoid the formation of blackish rings (in the case where the composition applied is black) and have good water resistance, while nevertheless being easy to remove, when compared with composition 3 (not containing any nonionic surfactant suitable for the invention) and composition 4 (not containing any particles of at least one polymer surface-stabilized with a stabilizer, dissolved in at least one hydrocarbon-based oil, which are suitable for the invention) not in accordance with the invention.

1. A composition for caring for and/or making up keratin fibres comprising at least:
a non-aqueous medium containing at least one hydrocarbon-based oil,
particles of at least one polymer that is surface-stabilized with a stabilizer, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4; and
a nonionic surfactant.

2. The composition according to claim 1, wherein said particles are in dispersion in said non-aqueous medium containing at least one hydrocarbon-based oil.

3. The composition according to claim 1, wherein the hydrocarbon-based oil(s) are present in a content ranging from 30% to 75% by weight relative to the total weight of the composition, the hydrocarbon-based oil(s) being apolar.

4. The composition according to claim 1, wherein the polymer particle(s) are present in an amount ranging from 5% to 40% by weight relative to the total weight of the composition.

5. The composition according to claim 1, wherein the polymer(s) of the particles are methyl acrylate and/or ethyl acrylate polymers.

6. The composition according to claim 1, wherein the polymer(s) of the particles comprise an ethylenically unsaturated acid monomer or the anhydride thereof.

7. The composition according to claim 1, wherein the polymer(s) of the particles comprise from 80% to 100% by weight of C₁-C₄ alkyl (meth)acrylate and from 0 to 20% by weight of ethylenically unsaturated acid monomer, relative to the total weight of the polymer, the polymer(s) of the particles being chosen from:
methyl acrylate homopolymers
ethyl acrylate homopolymers
methyl acrylate/ethyl acrylate copolymers
methyl acrylate/ethyl acrylate/acrylic acid copolymers
methyl acrylate/ethyl acrylate/maleic anhydride copolymers
methyl acrylate/acrylic acid copolymers
ethyl acrylate/acrylic acid copolymers
methyl acrylate/maleic anhydride copolymers
ethyl acrylate/maleic anhydride copolymers.

8. The composition according to claim 1, wherein the stabilizer(s) are statistical copolymers of isobornyl (meth)acrylate and of C₁-C₄ alkyl (meth)acrylate present in an isobornyl (meth)acrylate/C₁-C₄ alkyl (meth)acrylate weight ratio of greater than or equal to 5.

9. The composition according to claim 1, wherein the stabilizer(s) are chosen from:
isobornyl acrylate homopolymers
statistical copolymers of isobornyl acrylate/methyl acrylate
statistical copolymers of isobornyl acrylate/methyl acrylate/ethyl acrylate
statistical copolymers of isobornyl methacrylate/methyl acrylate.

10. The composition according to claim 1, wherein the combination of the stabilizer(s)+polymer(s) of the particles present in the dispersion comprises from 10% to 50% by weight of polymerized isobornyl (meth)acrylate and from 50% to 90% by weight of polymerized C₁-C₄ alkyl (meth)acrylate, relative to the total weight of the combination of the stabilizer(s)+polymer(s) of the particles.

11. The composition according to claim 1, wherein the composition comprises less than 10% by weight of water relative to the total weight of the composition.

12. The composition according to claim 1, wherein the nonionic surfactant(s) have an HLB value of greater than or equal to 8 at 25° C.

13. The composition according to claim 1, wherein the nonionic surfactant(s) are chosen from:
oxyethylenated ethers of glycerol;
oxyethylenated ethers of fatty alcohols;
fatty acid esters of polyethylene glycol;
esters of a fatty acid and of oxyethylenated glycerol ethers;
esters of a fatty acid and of oxyethylenated sorbitol ethers;
dimethicone copolyol;
dimethicone copolyol benzoate;
copolymers of propylene oxide and of ethylene oxide, also known as EO/PO polycondensates;
and mixtures thereof.

14. The composition according to claim 1, wherein the nonionic surfactant(s) are present in a content ranging from 1% to 15% by weight relative to the total weight of the composition.

15. The composition according to claim 1, wherein the composition further comprises at least one dyestuff.

16. The composition according to claim 1, further comprising at least one wax, the wax(es) being present in a content of greater than or equal to 5% by weight relative to the total weight of the composition.

17. The composition according to claim 1, further comprising at least one lipophilic gelling agent.

18. A cosmetic method for making up and/or caring for keratin fibres, comprising at least one step which consists in applying to said keratin fibres a composition comprising at least:
a non-aqueous medium containing at least one hydrocarbon-based oil,
particles of at least one polymer that is surface-stabilized with a stabilizer, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in isobornyl (meth)acrylate/C₁-C₄ alkyl (meth)acrylate weight ratio of greater than 4, and
a nonionic surfactant.

19. A process for preparing a mascara composition, wherein at least one stage of the process is carried out using a dispersion of particles of at least one polymer that is surface-stabilized with a stabilizer in a non-aqueous medium containing at least one hydrocarbon-based oil, the polymer of the particles being a C₁-C₄ alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4.
